(19) United States
(12) Patent Application Publication
(10) Pub. No.: US 2012/0320340 A1
(43) Pub. Date: Dec. 20, 2012

(54) SMART-PHONE ADAPTER FOR OPHTHALMOSCOPE

(75) Inventor: Wyche T. Coleman, III, Shreveport, LA (US)

(73) Assignee: INTUITIVE MEDICAL TECHNOLOGIES, LLC, Shreveport, LA (US)

(21) Appl. No.: 13/525,598

(22) Filed: Jun. 18, 2012

Related U.S. Application Data
(60) Provisional application No. 61/498,551, filed on Jun. 18, 2011.
(60) Provisional application No. 61/541,105, filed on Sep. 30, 2011.

(51) Int. Cl. A61B 3/15

(52) U.S. Cl. 351/208

(57) ABSTRACT

An adapter system connects a smart-phone or other camera to an ophthalmoscope or other viewing instrument at multiple locations with a single bracket. A fitting attached to the bracket connects the adapter to the viewing instrument in the region near its view port, close to the optical axis. A brace attached to the bracket connects the adapter to the viewing instrument in the region of the instrument's handle or other support structure, located away from the optical axis. The brace has a frame that holds the camera in place and aligns the camera lens with the optical axis of the instrument. The processor of the smart-phone or other mobile communications device can provide specific information related to the particular viewing instrument and can also be used in the operation and control of smart-scope instruments through a communications link.

### CROSS-REFERENCE TO RELATED APPLICATIONS

This application claims priority from U.S. Provisional Patent Application Nos. 61/498,551 and 61/541,105 as filed on Jun. 18, 2011 and Sep. 30, 2011 and which are incorporated by reference herein.

### STATEMENT REGARDING FEDERALLY SPONSORED RESEARCH

Not Applicable.

### APPENDIX

Not Applicable.

### BACKGROUND OF THE INVENTION

1. Field of the Invention

The present invention relates to ophthalmoscopes, and more particularly to adapters for holding an image capture device in an optical alignment with the eyepiece of the ophthalmoscopes and other viewing instruments.

2. Related Art

There are a number of hand-held ophthalmoscopes which include the ability to capture images of a patient's eye. Some of these hand-held instruments have included interchangeable instrument heads which can be used for a variety of medical examinations and could capture images of the patient's anatomy, either with still pictures or video. Most systems that had either been designed to work with an image capture device or that had been retrofitted to accept the image capture device used the standard lens fitting of a single-lens reflex (SLR) camera to secure the imaging device to the mating mount on the instrument's eyepiece. For SLR cameras, this traditionally had been a threaded screw-mount or a bayonet-mount.

As digital cameras became the standard for imaging technologies, a number of adapters were developed to hold the image capture devices in optical alignment with the eyepiece of the viewing instrument. Some adapters have even been designed for use with smart-phones that have their own camera features and functions. For those digital cameras that have a mounting platform for various lenses, some of the same adapters for SLR cameras or video cameras could be used. However, since many digital cameras and imaging smart-phones only have a single, integral lens system and no longer have any mounting device for different lenses, some adapters now secure the image capture device by its outer housing where a user would hold the device. At the same time, many of these adapters were still connected to the viewing instrument only at the eyepiece, and if there was another connection between the instrument and the camera, it was through a different bracket system that was not connected to the eyepiece. Since the lenses in many digital cameras, especially smart-phone devices, are offset from the center of gravity, the eyepiece connection is more likely to result in a misalignment between the optical axis of the camera and the optical axis of the instrument.

There have been some efforts to accommodate this misalignment, but they have been cumbersome, bulky and unwieldy. For example, the design described in U.S. Pat. No. 7,883,210 and US App. Pub. No. 2011/0085138 has an integrally constructed adapter with one portion substantially covering the ophthalmoscope as a housing, and the digital camera is substantially and not entirely housed in another portion of the adapter. In another example described in U.S. Pat. No. 7,465,049, the supporting part that surrounds the cellular phone has legs with engaging parts that fit into a dovetail groove. The supporting part is adjustable around the depth of the phone by support pillars in the legs and corresponding extendable fitting tubes which are biased with extension coil springs. Similarly, the supporting part is adjustable around the width of the phone by holding members which are also biased with an extension coil spring and are connected to the extendable fitting tubes. In these examples, the connection between the image capture device and the instrument is at or around the optical axis of the instrument and there is no portion of the bracket distal from the optical axis which is secured to the viewing instrument.

Generally, in adapters that connect cameras or other image capture devices to viewing instruments, there have been many variations in the arrangement of brackets, arms and other support structures which hold the cameras in place and align the cameras' lenses with the optical axis of the viewing instrument. However, none of the present systems describe or suggest an adapter which has a single bracket which is attached to both the view port of the viewing instrument through a fitting as well as the handle of the viewing instrument through a brace where this same bracket also has a frame that holds the camera in place so that its lens is aligned with the optical axis the viewing instrument.

### SUMMARY OF THE INVENTION

The present invention is an adapter system which connects an image capture device to an ophthalmoscope at multiple locations with one connection being proximate to the aligned optical axes of the image capture device and the ophthalmoscope and the other connection being distal to the aligned optical axes. In one aspect of the invention, the adapter has a bracket with a frame that holds the image capture device. The adapter has a fitting that is proximate to the lens of the imaging device, and the fitting connects to the ophthalmoscope's eyepiece. The adapter also has a brace that extends from a location on the imaging device which is distal to the lens (usually the grip portion of the imaging device) and connects to the ophthalmoscope's handle.

In another aspect of the invention, the adapter can be used with other types of medical viewing instruments as well as with other types of scopes in other industries. The adapter can be modular so that the bracket and frame can hold a particular type of camera, and the fitting for the eyepiece section of the bracket and the brace for the body section of the bracket can be selected for the particular scope or other viewing instrument to which the camera is to be coupled. In yet another aspect of the invention, the adapter system can be used with a smart-phone or other mobile communications device operating as the image capture device and the processor of such a device can be used to provide specific information related to the particular viewing instrument and can be used in the operation and control of smart-scope instruments through a communications link.

The present invention provides a novel design for an adapter that connects an image capture device to a viewing instrument with a single bracket that is attached to both the view port of the viewing instrument through a fitting as well as the handle of the viewing instrument through a brace. To ensure that the lens of the image capture device is aligned with the optical axis the viewing instrument, the bracket also has a frame that securely holds the camera in place.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The drawings constitute a part of this specification and include exemplary embodiments of the ophthalmological imaging apparatus, which may be embodied in various forms. It is to be understood that in some instances, various aspects of the invention may be shown exaggerated or enlarged to facilitate an understanding of the invention; therefore the drawings are not necessarily to scale. In addition, in the embodiments depicted herein, like reference numerals in the various drawings refer to identical or near identical structural elements.

FIG. 1 is an exploded perspective view of the adapter of the present invention with a viewing instrument and an image capture device.

FIGS. 2A-2D are alternate views of the adapter with a viewing instrument and an image capture device.

FIG. 3 is a perspective view of the adapter in as it is used with an ophthalmoscope and a smart-phone to image the back of a patient's eye.

FIG. 5 is a system diagram showing the adapter connecting an ophthalmoscope to a communications system through a smart-phone.

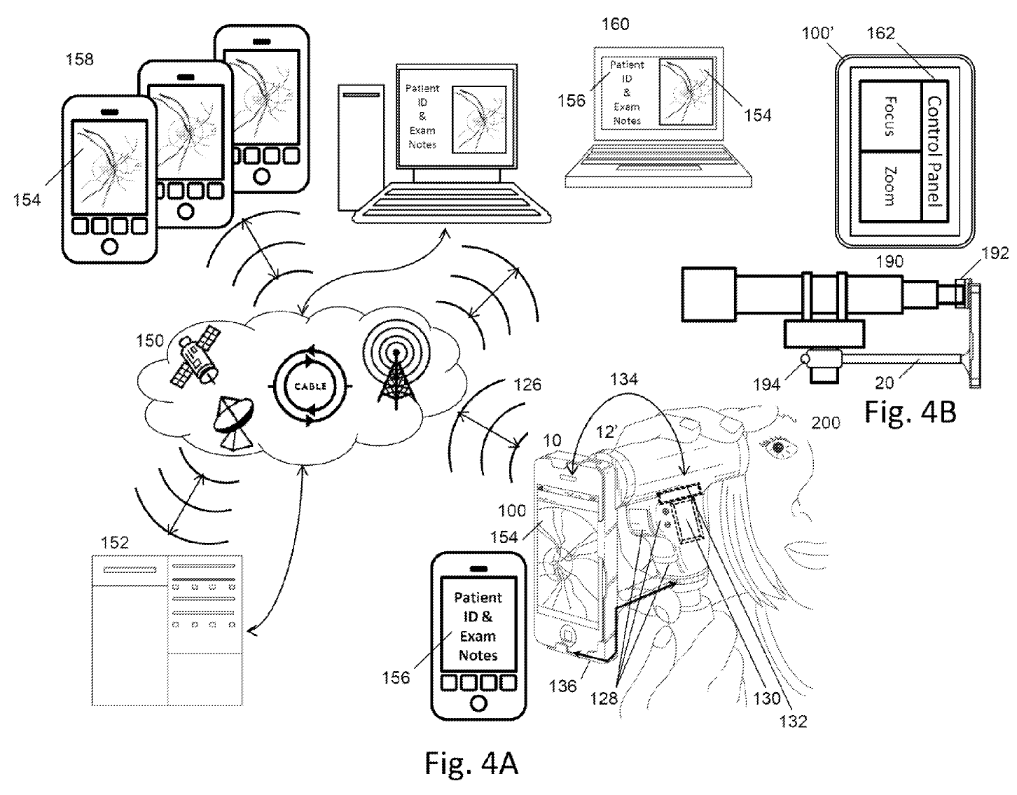

### DETAILED DESCRIPTION OF THE PREFERRED EMBODIMENTS

Described herein is an adapter 10 which holds a viewing instrument 12 in an aligned relationship with an imaging device that captures images of the subject being viewed. In the particular embodiment described herein, the viewing instrument is an ophthalmoscope 26 and the ophthalmological imaging apparatus allows a medical practitioner to easily view and record images of the retina and the anterior segments of the eye, including the optic nerve. Further, the ophthalmological imaging apparatus provides the medical practitioner with a complete view of the entire optic nerve, not just a partial view. Additionally, the ophthalmological imaging apparatus provides the medical practitioner with a full image of a patient's eye. Accordingly, it will be appreciated that the users of the present device can be a range of medical practitioners, such as optometrists, doctors, and nurses. It will also be appreciated that the preferred embodiment described herein is exemplary in nature, and as discussed below, the present invention can be applied to any viewing instrument 12, including any type of a medical scope as well as other types of scopes used by professionals in other industries or in education or any viewing instrument used by amateurs for sports and hobbies.

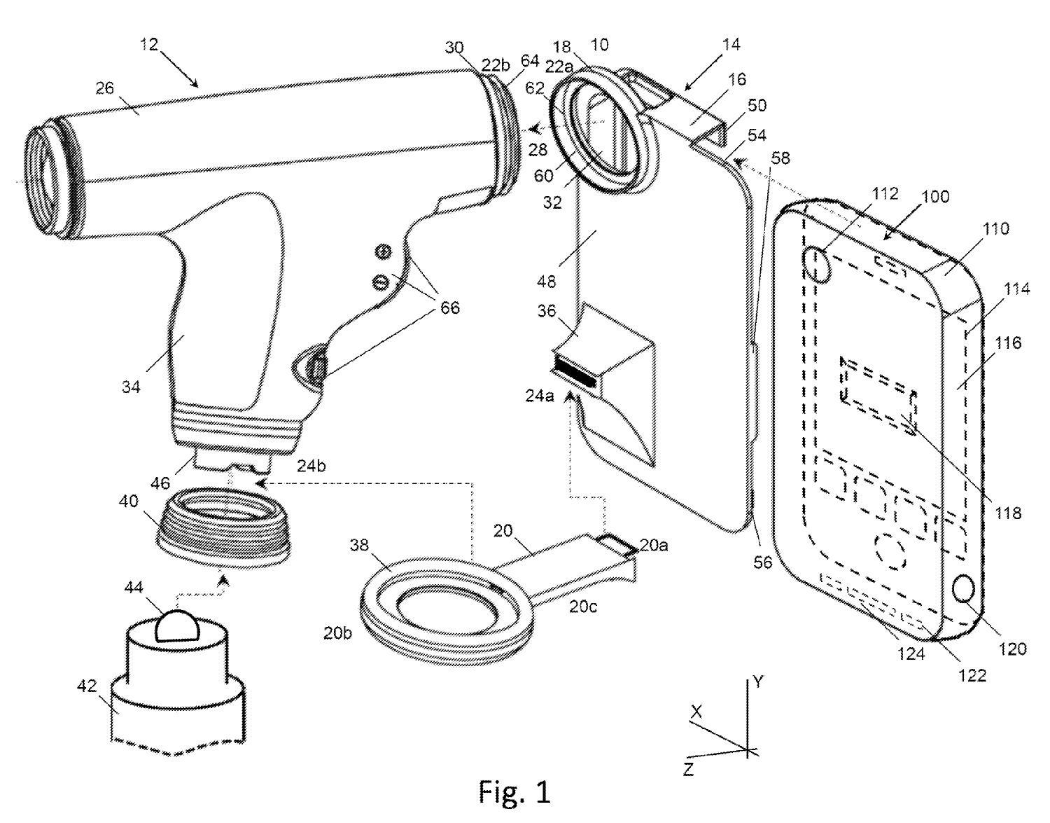

As shown in FIGS. 1-3, a camera or other image capture device 100 is connected to a viewing instrument 12 through an adapter 10. The adapter 10 has a bracket 14 which holds the imaging device in place within a frame 16. The adapter 10 also has a fitting 18 and a brace 20 which secure spaced-apart sections 22a, 24a on the bracket 14 to different corresponding locations 22b, 24b on the viewing instrument 12.

The image capture device could be any mobile communications device which has a camera system, preferably with a high resolution camera. Non-limiting examples of such mobile devices include: smart phones, such as an Apple iPhone® and the various smart phones using Google's™ Android™ operating system and other mobile communications devices, such as tablet computers and PDAs; digital cameras; and digital camcorders. In the preferred embodiment, the viewing instrument is an ophthalmoscope 26, such as a Welch Allyn PanOptic™ ophthalmoscope. Also, as indicated above, the viewing instrument 12 can be an ophthalmoscope or any other type of scope or viewing instrument. Accordingly, even though the preferred embodiments of the viewing instrument 12 and image capture device 100 are an ophthalmoscope 26 and a smart-phone 110 which may be referred to below as a scope and a camera, respectively, it will be appreciated that the adapter of the present invention can be applied to and used with any viewing instrument and image capture device.

In general, the present invention provides an improved adapter 10 that has a single bracket which holds the smart-phone 110 to the scope in different locations so that its camera lens 112 is aligned with the scope's optical axis 28 in a secure manner. The frame 16 of the bracket 14 holds the camera 112 in a set position in the x, y and z axes of the adapter 10. There are multiple connections between spaced-apart sections 22a, 24a of the same bracket and different corresponding locations 22b, 24b on the viewing instrument 110. The multiple connections are positioned at different sections of the camera to provide a more stable platform for the adapter than single-connection systems. Also, since the spaced apart connections are made through the same bracket, the adapter provides comparable stability as adapter systems which have multiple adapter brackets and are more complicated and bulky, such as adapters with one bracket that connects the base of the camera to the viewing instrument and another bracket that fits between the lens section of the camera and the viewing instrument.

With regard to the adapter 10 that connects the iPhone smart-phone 110 to the PanOptic™ ophthalmoscope 26 as particularly shown in FIGS. 1-3, the fitting 18 secures the eyepiece section 22a of the bracket to the scope proximal to the optical axis 28 in the region of the view port 30. The fitting surrounds a circular aperture 32 in the eyepiece section of the bracket. The brace 20 connects the body section 22b of the bracket to the scope distal from the view port and offset from the optical axis, such as at a handle portion 34 of the scope. The brace 20 preferably extends to its distal end 20a at the handle from its proximal end 20b at a platform mount 36 on the bracket through an arm 20c which has an axis that is substantially in a plane with and offset from the ophthalmoscope's optical axis. The distal end 20a preferably includes a locking ring 38 that is secured to the lower end of the handle by a nut 40 that fits with corresponding threads at the base of the handle. The lower connection that is distal from the optical axis provides rigidity to the overall adapter and helps keep the adapter properly aligned with the ophthalmoscope, including alignment in the z-axis along the optical axis. A battery pack 42 with a light bulb 44 has a top end which extends through the nut and secures to the handle with a bayonet mount 46.

In the preferred embodiment, the brace is removably connected to the bracket through the platform mount, but in an alternative embodiment, the brace may be permanently fixed to the bracket. Also, in the preferred embodiment, the components in the eyepiece section and body section of the bracket are integrally formed with the frame. It will also be appreciated that some components of the adapter can be manufactured separately and then permanently connected through means known to one skilled in the art, including but not limited to, welding, glue, or epoxy. The adapter can be made of metal, alloys, hardened rubber, plastic, or any combination thereof.

Each one of the means for removably attaching or otherwise connecting or securing the viewing instrument and the image capture device to the same bracket may be any means known to one skilled in the art. Such connections include but are not limited to, a snap-fit mount, a screw-mount, a ring-lock clamp, a bayonet-mount, slotted tabs and sliding slots or grooves, hook and loop fasteners, a friction fit attachment and possibly even an annular ring with set screws through the ring and other forms of clamps. As described in more detail below, the connection between the view port and the fitting is an example of a snap-fit connection, and the holding of the smart-phone in the frame is an example of friction fit connections. The connection between the proximal end of the brace and the platform mount is an example of a sliding interlock connection (t-slot or dovetail groove). The distal end of the brace is an example of a locking ring connection with a threaded screw-mount clamp on the handle, and the handle also has a bayonet mount for the battery pack.

As particularly shown in FIG. 1, the bracket 14 has a panel 48 attached to the frame's front side 16a. The panel preferably has a substantially planar surface that extends between the fitting 18 and the platform mount 36, and the length of the panel section between the fitting and platform mount is greater than the diameter of the aperture 32. In the preferred embodiment, the length of this panel section is equal to or longer then the length of the brace's arm.

As indicated above, the frame 16 is sized to hold the smart-phone in a friction fit manner. In particular, the frame has one or more lips 50 on its back side 16b opposite to the panel. The frame also has a pair of opposing end rails 52a and a side rail 52b extending between and connecting the end rails. The frame is preferably a slightly undersized case with one side not having a side rail so that the smart-phone can slide into the frame with the grip portion 116 of the image capture device fitting between the lips on the back side and the panel section on the front side. The lips 50 preferably extend from the end rails and the side rail. On the side of the frame without a side rail, opposite the side rail, the panel has an extension 54 beyond the end rails. The panel extension 54 preferably includes an edge 56 opposite the side rail and a ridge 58 proximate to the edge. The ridge extends to engage the grip portion 116 of the image capture device.

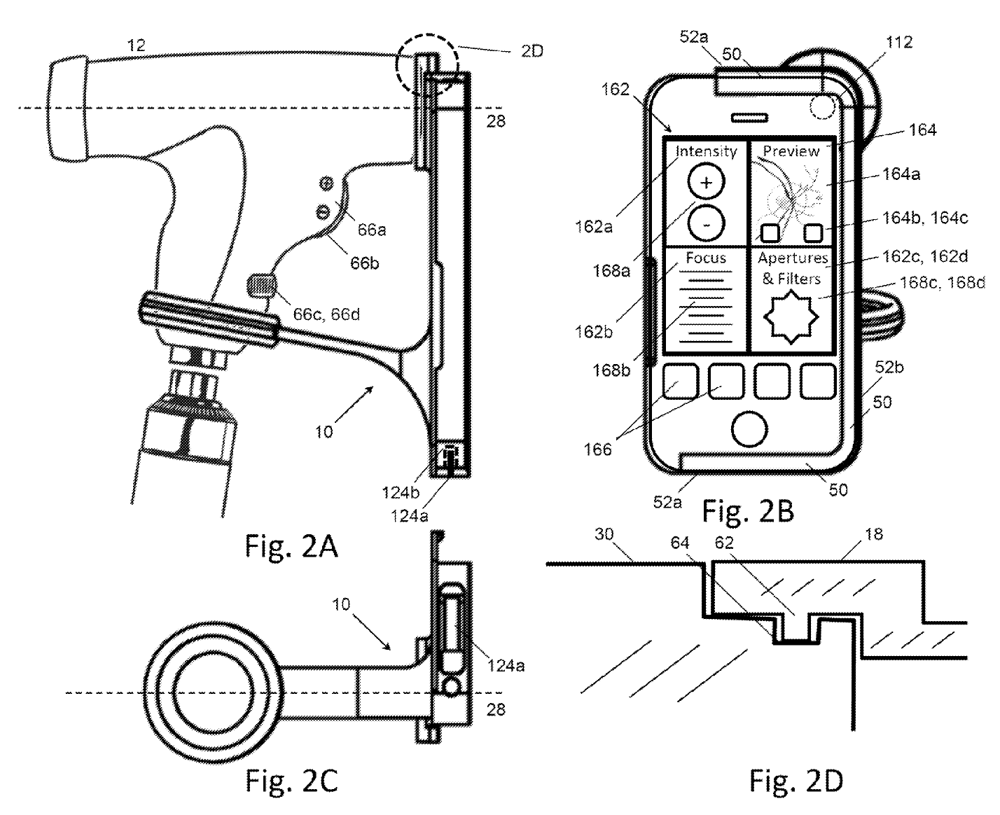

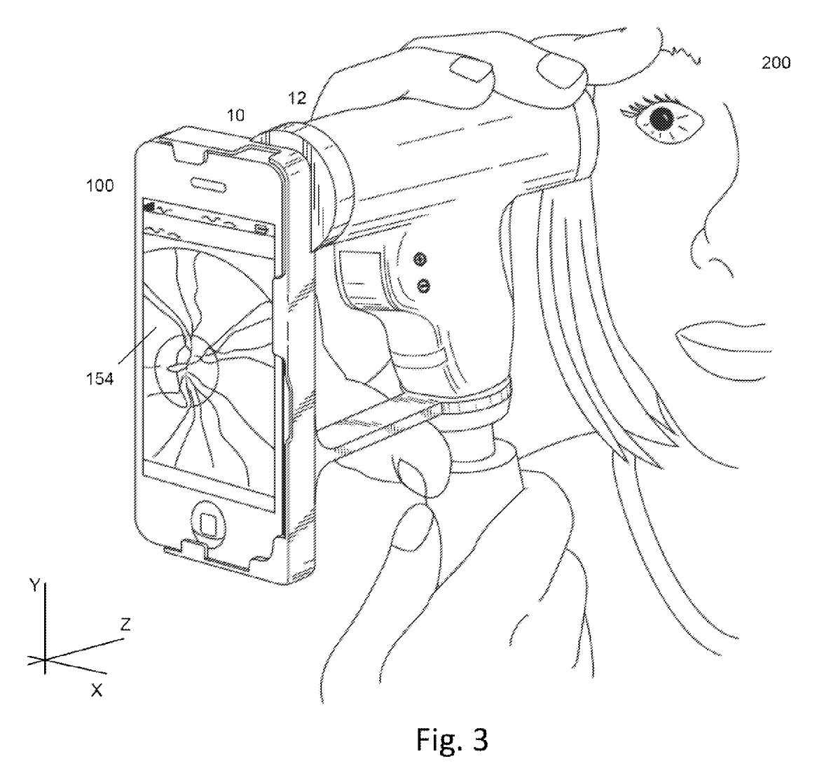

As particularly shown in FIGS. 2B and 3, the display screen 114 of the image capture device is displayed substantially within the frame and with substantially no blockage of the screen by the lips 50. In FIG. 2B, the lips extend along substantially the entirety of the side rail and end rails. In the alternative embodiment shown in FIG. 3, the lips 50 are tabs on a portion of the side rail and on the top and bottom end rails as well as at the bottom corner of the frame between the side rail and the bottom end rail.

The view port of the ophthalmoscope and many other scopes typically have a side brow rest with a soft rubber cover to shield the viewer's eye from ambient light when peering through the scope. The rests and/or covers may be connected to the scopes by a snap-fit or screw-in arrangement, and since they are not necessary when using the imaging device, the adapter can use these connection elements to secure the eyepiece section of the bracket to the scope. Generally, the fitting 18 has a wall 60 that extends beyond the bracket and covers the view port of the viewing instrument. The wall has a circumference 60a which partially extends outside the frame and a center 60b inside the frame.

The preferred snap-fit connection of the bracket's eyepiece section is shown in FIG. 2A, and FIG. 2D shows a detail view of a ring 62 in the fitting which mates with the annular groove 64 in the scope's view port. In the preferred embodiment, the snap-fit version of the fitting is integrally formed with the bracket. For other fittings that do not have a snap-fit connection, it may be preferable for one or more features of the fitting to be separate from the bracket. For example, in a threaded screw mount fitting, a base portion of the fitting may be integrally formed with the bracket, but the screw portion of the fitting would rotate around the base and would not be integrally formed with the bracket.

With the adapter secured to the scope, the frame holds the smart-phone to the bracket so that the camera is positioned in the x, y and z axes with optimal optics. It will be appreciated that the means for attaching the smart-phone to the bracket can be any attachment means known to those skilled in the art, including but not limited to, a pair of arms located on opposite sides of the bracket's body section for slidable engagement with the smart-phone, a pair of arms located at opposite ends of the bracket for slidable engagement of the smart-phone, a case formed with and permanently attached to the bracket so that the case houses the smart-phone, a snap-fitting arrangement wherein a snap-fit is secured to the bracket, and a slidable engagement wherein the mobile imaging device slides into a fitting arrangement with an element located on the first frame member.

The adapter can be modular with brackets being designed to hold particular image capture devices. The fitting for the eyepiece section of the bracket and the brace for the body section of the bracket can be selected for the particular viewing instrument to which the image capture device is to be coupled. For example, as shown on FIG. 4, an alternative brace could be used to secure the body section of the bracket to a telescope 190. The frame of the bracket for holding the smart-phone could be the same as described above for the ophthalmoscope. The fitting may have a snap-fit connector 192 which secures the eyepiece section of the bracket to the particular type of view port on the telescope. For example, as discussed above, the snap-fit connector could attach a threaded screw mount to the bracket. Also, the distal end of the brace may have a different type of attachment structure, such as a tube clamp 194 (or a hose clamp).

In addition to the ophthalmoscope 26 and the telescope 190, the adapter can be modified so that other types of viewing instruments 12 can be connected to various types of smart-phones and other cameras. Generally, the adapter can be used for medical imaging devices to facilitate the viewing and capturing of images of a patient's ear, nose, throat or other anatomical feature. Accordingly, the present invention can be used with any medical viewing instrument, such as an endoscope, an otoscope, a dermatoscope, a laryngoscope, a laparoscope and any other medical instrument that is used to view a patient's internal or external anatomy. Additionally, the adapter of the present invention can be further modified and used with other viewing instruments that are used in industry and education or for sports and hobbies, such as the telescope 190 described above or a microscope, a borescope or even a sighting scope, a surveyor scope or binoculars.

FIG. 3 shows the ophthalmic imaging apparatus embodiment of the present invention as it is used in operation with a subject patient 200. The ophthalmological imaging apparatus allows the mobile imaging device to be in optical communication with an ophthalmoscope. The term “in optical communication” means that two or more devices perceive the same image. For example, a smart-phone in optical communication with the scope head of an ophthalmoscope is capable of capturing the image as viewed by the ophthalmoscope. The ophthalmological imaging apparatus allows a user to view the retina and anterior segment of an eye without the difficulty of aligning both the user's eye and the patient's eye with the ophthalmoscope head. Further, the ophthalmological imaging apparatus provides for a firm and stable mounting of the mobile imaging device to the scope head of an ophthalmoscope at a well functioning angle for examining a patient's eye.

In one embodiment, the mobile imaging device can take still images. In another embodiment, the mobile imaging device may record video images. In yet another embodiment, the mobile imaging device is capable of taking still images and video images. In a further embodiment, a mobile imaging device capable of recording video images may record audio along with video images. Once removably attached to the adapter for the ophthalmoscope, the imaging device is able to capture images of the eye, including images of the retina and optic nerve. The image captured on the mobile imaging device is the same view a practitioner would see when traditionally using an ophthalmoscope. The images may be viewed and analyzed either on the mobile imaging device or on a computer, as described below.

While the present invention is particularly described with reference to the adapter as it is connected to the ophthalmoscope or the telescope, it will be appreciated that the adapter could also be used to connect a smart-phone to an analog camera which may have better telephoto capabilities and optics than the smart-phone. Also, many digital cameras do not have any communications link to transmit images over a communications network, and in some cases, it may be preferred to physically connect a smart-phone to a digital camera in order to provide immediate communications over the communications network through the smart-phone's communications link rather than first capturing the still images or video on the digital camera and then transferring the image file(s) to a local computer or the smart-phone and then sending the files to the communications network through the communications link of the local computer or the smart phone.

Many viewing instruments with excellent optics do not have the capability to capture images or communicate these images, such as the PanOptic™ ophthalmoscope described above. The adapter of the present invention combines smart-phone technology with these viewing instruments. In particular the smart-phone's computer processor 118 allows the user to communicate captured images 154 over a communications network 150, such as shown in FIG. 4. The apparatus can be linked with a centralized server 152 through the network 150 to send the captured images 154 and other information 156. As discussed in detail below, the Also, a doctor can send the captured images directly to the smart-phones 158 or computers 160 of other doctors. In a medical practice or hospital system, these other doctors may be given access to the captured images and information stored on the centralized server.

The smart-phone's processor 118 can also run an examination application that can be used for taking examination records and notes 156, annotating, processing and storing the captured images 154, and for sharing the images and corresponding information with the other available resources through the communications network 150. The smart-phone could also use the microphone 120 and speaker 122 for taking and listening to voice notes, which may include capturing and replaying audio recordings with video recordings being made of the subject 200.

The use of the ophthalmological imaging apparatus according to the present invention allows a user to capture images of a patient's eye in out-of-office settings when it would be impractical for the user to transport traditional equipment capable of viewing and photographing an eye. The ophthalmological imaging apparatus allows the user to communicate with medical specialists, such as optometrists and ophthalmologists, who can be in another location yet help diagnose and evaluate a patient's eye. For example, an emergency room physician may use the ophthalmological imaging apparatus to send images of a patient's eye to an ophthalmologist to diagnose an injury to the patient's eye. The images may be transmitted to specialists with other computer tools that can further analyze the captured images.

In one embodiment, the images may be transferred to a network accessible by physicians for analysis. In a further embodiment, the mobile imaging device communicates images and patient records and information to a network of physicians and ophthalmologists for second opinions and assistance in diagnosing issues. In still another embodiment, the transfer of the images to the network may be through a wireless transfer of the images directly from the mobile imaging device to the network. In an alternate embodiment, the images are transferred from the mobile imaging device to a computer and then once on the computer, the images are transferred to the network of physicians. In yet another embodiment, the network also includes patient data and patient medical history to aid in the physician's analysis of the images.

When running an examination application, the smart-phone processor can help the doctor optimize the use of the ophthalmoscope by using the scope's image control features 66, such as the light intensity setting 66a, the focus wheel setting 66b, the aperture setting 66c, and the filter setting 66d. As described in detail below with reference to FIGS. 2B and 4, the combination of the smart-phone with the ophthalmoscope through the adapter results in an integrated ophthalmic viewing and examination system. In functioning as an integrated system, the computer processor produces one or more control panel displays 162 on the screen which correspond with the scope's image control features as well as an image preview 164 which shows live subject views 164a from the viewing instrument. The control panel displays may be shown on a single screen, and button selectors 166 can be used to select a control panel display for its own screen.

The control panel displays 162 for the image control features 66 preferably have one or more image control selectors 168 corresponding with particular image control features used with the viewing instrument. For example, as shown on FIG. 2, the ophthalmoscope control panel displays could include a light intensity display 162a, a focus display 162b, an aperture display 162c, and/or a filter display 162d which respectively correspond with the light intensity setting 66a, the focus wheel setting 66b, the aperture setting 66c, and the filter setting 66d on the scope. It will be appreciated that the image control features could vary depending on the type of viewing instrument that is mated with the smart-phone. For example, control panels for a telescope 190 could also have a focus display and may have a magnification display as well.

As indicated above and particularly shown in FIG. 4, it is possible to resize the adapter for use with other types of mobile communication devices that have integral camera systems, such as a tablet computer 100′ or even digital cameras. A tablet computer may be more desirable for certain uses, such as with the telescope 190 in which a larger display may be more desirable, or for certain users, such as with academic instructors in educational environments where the computer systems may already have tablet computers in the laboratories. Of course, the orientation of the tablet computer can be vertical or horizontal, as can the orientation of most smart-phones, and adapters can be made to accommodate either orientation. Such modifications to the size and orientation of the adapter for various mobile communications devices are a matter of design choice, and it should be appreciated that indications of particular mobile communications devices, such as a smart-phone, a tablet computer or a digital camera, is an exemplary description and is not limiting to the scope of this invention.

The image preview screen 164 could also include one or more capture image commands 164b, 164c for the image capture device to produce a captured image 154 from the live subject views 164a. In addition to viewing the subject images on the display of the smart-phone, one of the capture image commands 164b could instruct the processor 118 to capture of multiple images in series over a several second period to give the clinician the best chance possible of obtaining quality images. This option frees the clinician from the burden of having to interact with the smart-phone device while simultaneously trying to align the ophthalmoscope with the desired area of the retina to be viewed. The clinician may attempt to view the optic nerve for six (6) seconds, but only obtain a satisfactory view for one-half of a second. The processor can also allow the user to select the frames during the period in which the system produced good images.

Other capture image commands 164c could be used to select or adjust a countdown time before the recording starts and/or adjust a preset recording time for capturing video images. The processor can provide the user with default times, such as a countdown time of three (3) seconds and a recording time of five (5) seconds. The user can review images on the screen in real time and can replay the video and/or scroll through a series of freeze-frame images. The user can select one or more images and store high resolution version of the selected image.

For scopes that have analog controls without any communication of the scope settings on each of the image control features, the user may enter one or more scope settings into the image control selectors 168a, 168b, 168c, 168d for the corresponding image control features. As explained below, the user can provide an instruction to the smart-phone's processor to perform one or more tasks with the scope settings information that is entered into the smart-phone. One of the button selectors could be used to enter the instruction or the control panel display for one or more of the image control selectors 168 could have a section for entering or selecting the task instruction. One of the button selectors 166 could be used to indicate a smart-scope selection for one or more of the image control selectors. As described in detail below, smart-scopes can automatically communicate scope settings to the smart-phone and may permit the processor to perform even more tasks.

The smart-phone can perform a number of processing functions with the scope settings information. For example, the processor can determine a focusing adjustment in the live subject views and can determine a refractive error from the live subject views. The processor can also calculate a differential power level between a focused image and an unfocused image and can determine a calibration factor for optimizing the live subject views. As described above, the processor can also perform a time countdown before starting to record the live subject views as captured images.

In the particular example of calibrating an ophthalmoscope, a doctor can enter a particular setting of the focus wheel 66a into the control panel for the focus image control selector 168b. The doctor can enter information to the processor that the setting corresponds with an emetropic human eye so that the ophthalmoscope is calibrated to a standardized setting. The processor can also perform an initial analysis of the eye. For example, the system can be optimized for the combination of a particular type of smart-phone that the adapter connects to a particular type of ophthalmoscope that is calibrated to a particular refractive index. The smart-phone's camera lens could be used to adjust the focus and the processor could calculate the refractive error of the subject's eye based on the amount of focal power that is required by the camera system's focusing lens to obtain the focused image. The processor could be used to calibrate the system, whereby the camera's focusing lens uses a particular amount of focal power to obtain a clear image of an emetropic human eye. By knowing this baseline level of power that is required for focusing the image of an emetropic human eye, measuring the power level that is required to focus on a particular subject's eye, and having a correlation between the differential power levels and refractive errors of the subject's eye (myope and hyperope), the processor can determine the refractive error. Also, the processor may be able to analyze the images that are captured using the ophthalmological imaging system.

It will be appreciated that this system for allowing the smart-phone's camera lens to focus the image received through the viewing instrument set at a calibrated focal power and processing this information could be useful in with a variety of viewing instruments and not just an ophthalmoscope. For example, with the telescope, the focal power information may be used in determining distances to an object or between multiple objects that can be alternatively selected within the field of view of the smart-phone's camera lens.

Regardless of the viewing instrument being used, the captured images and other information can be communicated to a computer using the communication system of the image capture device. In some cases, communication is made through a physical cable, such as a USB cable. In the preferred embodiment, the communication is made through a wireless transmission 126 from the smart-phone to the communications network 150 or a local computer by methods known to those skilled in the art.

The viewing instrument 12 can also have sensors 128 that measure the settings of the image control features 66 as well as its own computer processor 130 and communications module 132. A viewing instrument with these features can be generally referred to as a smart-viewing device 12′ or a smart-scope. The measured settings may be processed by the smart-scope's processor and may be used in a feedback system with setting controllers (not shown) that can adjust the image control features in response to a selection made by a user. Such controllers can include servos and other motors that are generally know, such as auto-focusing systems and auto-shutter light adjustments in cameras. The communications module 130 transmits the measured settings to the smart-phone's computer processor 118.

It will be appreciated that in transmitting the measured settings, the communications module 132 may use a wireless communications link 134 with the smart-phone 100, such as a Bluetooth® wireless communication link, and may also have a communication port that can either receive a cable or a direct communications interface 136 through the adapter 10. Such a physical communications interface between a smart-scope and a smart-phone is preferably made in the body location rather than the view port location, and the corresponding section of the frame 16 could include a docking connector 124a that interfaces with the phone's docking port 124b. With a docking connector on the bottom end rail, a lip may not be required at the bottom of the frame and may only be needed on the side rail and possibly in the top corner at the intersection of the side rail and the top end rail.

Even though it is possible to incorporate the processing functions described above into the smart-scope's processor 130, it is preferred that these processing functions be incorporated into the smart-phone's processor 118. By incorporating these processing functions into a general purpose smart-phone, tablet computer or other mobile communications device with an integral camera system and computing capabilities, the cost of the overall system will be less because of the economies of scale in using modular elements. Additionally, with a general purpose smart-phone, the processing software can be updated as the computing power and storage capacity of these devices continue to increase. Updates to the software may include additional processing functions and additional analyses of the images, such as pattern recognition evaluations and tools, which cannot be efficiently performed on current smart phones. Yet another benefit of the modular nature of the present invention is the increasing resolution and imaging power of smart-phones. The optics of the viewing instruments, such as ophthalmoscopes, may already be optimized, and being able to swap in new smart-phones with better computing and imaging capabilities will continue to improve the overall system without having to replace those elements in the system that are already optimized. This will further reduce the overall operating costs of the system over the lifespan of each component because the user will be able to replace each component individually as it reaches the end of its life rather than replacing the entire system.

With two-way communications between the smart-phone and the smart-scope, the smart-phone processor can use either one of the communications links 134, 136 to transmit controller information to smart-scopes that have setting controllers. In this embodiment of the system, a user would be presented with setting selection options on the smart-phone's control panels and the smart-phone would receive setting selection entries from the user through the control panels. It will be appreciated that smart-phones with voice recognition technology can accept a user's voice command to change control panels and or to select options that are on the active control panel or may even be on another control panel. When a change is made to a setting according to a user's selection or other command or instruction and it is transmitted to the smart-scope, the settings controller in the smart-scope changes the scope setting for the particular image control feature and the smart-scope sends back the new setting information to the smart-phone.

According to the ophthalmologic imaging embodiment of the present invention, the interface between the smart-phone and the ophthalmoscope includes the data exchange between the two devices. The exchange of data is preferably used to document and add to the patient file as well as to process the internal settings of the ophthalmoscope, such as the refractive power used to obtain a clear image. As discussed above, with an ophthalmoscope that has setting controllers for the image control features 66, the smart-phone can control functions of the ophthalmoscope such as the refractive power setting, light output level color and size, and may even provide a trigger for an increased light level for the purpose of flash photography.

Even though it is easier to pass information between the smart-scope version of the ophthalmoscope and the smart-phone, as indicated above, a user may read certain settings an analog ophthalmoscope, such as the PanOptic ophthalmoscope, and enter the information into the smart-phone's processor through the control panels. Accordingly, regardless of whether the viewing instrument is a standard analog scope or a smart-scope, the smart-phone can use its own internal camera system change the overall refractive power of the ophthalmologic imaging system in order to automatically capture clearly focused images.

With an analog ophthalmoscope, the focal power of the scope's lens system can be calibrated so that it causes the focusing lens within smart-phone camera (material that changes refraction index as voltage is applied) to use approximately one-half of its focal power to obtain a clear image of an emetropic human eye (i.e., no refractive error in the eye). This configuration would allow the smart-phone camera to automatically add or subtract focal power and enable the clear imaging through a wide range of refractive errors in the lens of the subject's eye (myope and hyperope) with no additional focusing lenses required in the adapter. By calculating the focal power required by the smart-phone's camera system to obtain a clear image, an approximation of refractive power of the subject eye can be determined. It will be appreciated that the scope could also be calibrated with the smart-phone's camera system for several different viewing options, such as a standard field of view through non-dilated pupil and a wider field of view through a dilated pupil. Of course, with the smart-scope, the smart-phone may send signals to the ophthalmoscope to change the refractive power, and in this case the smart-phone would be able to directly calculate the refractive power of the subject eye based on the measured settings from the smart-scope and corresponding optics tables for the smart-scope's lens positions.

It will be appreciated that the present invention for the ophthalmological imaging system provides non-eye specialists with a method of non-mydriatic or mydriatic fundus photography at an extremely low cost. More generally, for viewing instruments generally, since smart-phones have their own computer processors and displays that can be used to control various functions of the smart-phone, such as the communications module and the camera system, one or more specialized computer applications running on the smart-phones can serve as the control panel for optimizing the use of the smart-phone with a viewing instrument which may be an analog scope or a smart-scope.

As indicated above, the smart-phone can be adjusted for a range of scope settings. For example, with ophthalmoscopes in particular, red filters may be used to decrease the percentage of color spectrum received that is in the red spectrum. This would increase image contrast while imaging the retina, as it is mostly pigmented red. Also, it is often difficult for a clinician to obtain a good view of the retina through an undilated pupil for long periods of time which would typically be required for good photography. This is partially due to low patient tolerance for bright lights and limited ability to hold the ophthalmoscope perfectly steady. The latter half of this problem can be mitigated by the ability of the clinician to look at the live subject images on the smart-phone's display screen rather than having to press their own eyes against the view port of ophthalmoscopes and fundus cameras. With regard to patient tolerance of light, when the smart-phone is used with a smart-scope, the processor could reduce the light intensity while the clinician aligns the device with the portion of fundus that is to be imaged and then increases the intensity when the clinician selects the capture image command. This could allow focusing of the camera lens with better patient tolerance. Of course, with auto-focus capabilities in either a smart-scope ophthalmoscope or in the smart-phone, the time to focus the overall ophthalmologic imaging system could be greatly reduced. The processor can also optimize the use of the particular ophthalmoscope optics with the features of the smart-phone.

For the purpose of understanding the present invention, references are made in the text to exemplary embodiments of an ophthalmoscope and a telescope. It should be understood that no limitations on the scope of the invention are intended by describing these exemplary embodiments. The adapter of the present invention can connect any image capture device to a viewing instrument using any attachment means that has a single bracket with a frame that holds the image capture device securely in place, and the same bracket has an eyepiece section and a body section which respectively connect the lens of the image capture device proximate to the view port section of the viewing instrument and the a distal portion of the image capture device to another section of the viewing instrument, such as a handle or support. The adapter aligns the image capture device's camera lens, preferably a high resolution camera system, with the optical axis of the viewing instrument. With the image capture device mated to the viewing instrument with the adapter, a processor that is preferably in the image capture device can be used to display control panels and preview screens, receive information from the user or a smart-scope and perform a number of processing tasks that improve the overall imaging system. For the ophthalmological imaging embodiment, the adapter securely aligns the optical axis of the ophthalmoscope and the smart-phone in the x-axis, the y-axis and the z-axis.

One of ordinary skill in the art will readily appreciate that alternate but functionally equivalent components, materials, designs, and equipment may be used, particularly including other viewing instruments and smart-viewing devices. Specific elements disclosed herein are not to be interpreted as limiting, but rather as a basis for the claims and as a representative basis for teaching one of ordinary skill in the art to employ the present invention. Accordingly, the above description is intended to cover such alternatives, modifications, and equivalents as may be included within the spirit and scope of the invention as defined by the appended claims. Additionally, the terms “substantially” or “approximately” as used herein may be applied to modify any quantitative representation that could permissibly vary without resulting in a change to the basic function to which it is related.

What is claimed is:

1. An adapter for connecting an image capture device to a viewing instrument, wherein the image capture device has a camera lens, a screen and a grip portion and the viewing instrument has a view port, an optical axis and a handle, comprising:
a bracket comprising a frame and an aperture, wherein said frame at least partially encloses the grip portion of the image capture device and said aperture is located within said frame, and wherein said frame removably holds the image capture device in a fixed position in said bracket with the camera lens aligned with said aperture;
a fitting attached to said bracket and surrounding at least a portion of said aperture in said bracket, wherein said fitting secures said bracket to the viewing instrument proximal to the view port and aligns said aperture with the view port at the optical axis of the viewing instrument; and
a brace having a proximal end, a distal end and a support arm extending between said proximal end and said distal end, wherein said proximal end is connected to said bracket at a position offset from said aperture and wherein said distal end is fixed to the viewing instrument.

2. The adapter of claim 1, wherein said bracket further comprises a panel attached to a front side of said frame proximate to the viewing instrument, wherein a back side of said frame comprises a lip opposite said panel, wherein the grip portion of the image capture device fits between said lip and said panel, and wherein the screen of the image capture device is displayed substantially within said frame and with substantially no blockage of the screen by said lip.

3. The adapter of claim 2, wherein said frame further comprises a plurality of lips on said back side, a pair of opposing end rails, and a side rail extending between end rails, wherein said lips extend from said end rails and from said side rail, wherein said panel is comprised of a substantially planar surface with an extension beyond at least one of said end rails away from said side rail, wherein said panel extension further comprises an edge opposite said side rail and a ridge proximate to said edge, said ridge extending to engage the grip portion of the image capture device.

4. The adapter of claim 1, wherein said fitting comprises a wall extending beyond said bracket and covering the view port of the viewing instrument, said wall comprising a circumference partially extending outside said frame and a center inside said frame.

5. The adapter of claim 4, wherein said fitting is integrally formed with said bracket as a snap-fit mount.

6. The adapter of claim 1, wherein said brace is offset from said aperture by a distance at least as great as a diameter of said fitting.

7. The adapter of claim 1, wherein said bracket further comprises a platform mount slidably receiving said proximal end of said brace and wherein said distal end of said brace further comprises a locking ring secured to the handle of the viewing instrument.

8. An adapter for connecting an image capture device to a viewing instrument, wherein the image capture device has a camera lens, a view section and a grip portion and the viewing instrument has a view port, an optical axis and a housing, comprising:
a bracket comprising a frame, an aperture and a panel section extending between and connecting said frame and said aperture, wherein said bracket has a body section and an eyepiece section, wherein said frame engages the grip portion of the image capture device at said body section of said bracket and removably holds the image capture device in a fixed position in said bracket with the camera lens aligned with said aperture, wherein said aperture has a diameter and is located at said eyepiece section of said bracket proximal to the viewport of the image capture device, and wherein said panel section has a length between said body section and said eyepiece section within said frame, said length being greater than said diameter of said aperture;
a fitting attached to said bracket and surrounding at least a portion of said aperture in said bracket, wherein said fitting secures said eyepiece section of said bracket to the viewing instrument proximal to the view port and aligns said aperture with the view port at the optical axis of the viewing instrument; and
a brace having a proximal end, a distal end and a support arm extending between and connecting said proximal end and said distal end, wherein said proximal end is connected to said body section of said bracket and wherein said distal end is fixed to the viewing instrument at a position offset from the optical axis.

9. The adapter of claim 8, wherein said frame comprises a pair of opposing end rails, a side rail extending between end rails, a front side, a back side, and a plurality of lips extending from said end rails and from said side rail on said back side, wherein said frame further engages said view section of the image capture device at said eyepiece section and the view section is displayed within said frame with substantially no blockage of the view section by said lips, wherein said panel is attached to said front side and is comprised of a substantially planar surface with an extension beyond at least one of said end rails away from said side rail, wherein the grip portion of the image capture device fits within said end rails and side rail between said lips and said panel, and wherein said panel extension further comprises an edge opposite said side rail and a ridge proximate to said edge, said ridge engaging the grip portion of the image capture device.

10. The adapter of claim 9, wherein said fitting further comprises a wall extending from said bracket and covering the view port of the viewing instrument, said wall comprising a circumference partially extending outside said frame, wherein said bracket further comprises a platform mount to which said proximal end of said brace is removably attached, wherein said distal end of said brace further comprises a locking ring secured to the housing of the viewing instrument and wherein a distance between said fitting and said platform mount is equal to or greater than a length of said arm.

11. A viewing system for an image capture device, wherein the image capture device has a camera lens, a screen and a grip portion, comprising:
a viewing instrument comprising a view port, a housing, an optical axis within said housing and through said view port and a support structure connected to said housing and extending away from said optical axis; and
an adapter connected to said viewing instrument at a first location proximal to said view port and at a second location proximal to said support structure, said adapter comprising a bracket, a fitting and a brace, wherein said bracket is comprised of a frame and an aperture, wherein said frame removably holds the image capture device in a fixed position in said bracket with the camera lens aligned with said aperture, wherein said fitting is attached to said bracket and surrounds at least a portion of said aperture in said bracket, wherein said fitting secures said bracket to the viewing instrument proximal to the view port and aligns said aperture with the view port at the optical axis of the viewing instrument, wherein said brace comprises a proximal end, a distal end and a support arm extending between said proximal end and said distal end, wherein said proximal end is connected to said bracket at a position offset from said aperture and wherein said distal end is fixed to the viewing instrument at the support structure.

12. The viewing system of claim 11, wherein said viewing instrument is selected from the group of instrument types consisting of an ophthalmoscope, an endoscope, an otoscope, a dermatoscope, a laryngoscope, a laparoscope, a telescope, a microscope, a borescope, sighting scope, a surveyor scope and binoculars, and wherein said support structure is selected from the structural group consisting of a handle, a stand, a gantry, a tripod, and any combination thereof.

13. The viewing system of claim 11, wherein said bracket further comprises a panel and said frame comprises a pair of opposing end rails, a side rail extending between end rails, a front side, a back side, and a plurality of lips extending from said end rails and from said side rail on said back side, wherein said panel is attached to said front side and is comprised of a substantially planar surface with an extension beyond at least one of said end rails away from said side rail, wherein the grip portion of the image capture device fits within said end rails and side rail between said lips and said panel, wherein the screen is displayed substantially within said frame and with substantially no blockage of the screen by said lips, wherein said fitting comprises a wall extending beyond said bracket and covering the view port of the viewing instrument, said wall comprising a circumference partially extending outside said frame and a center within said frame.

14. The viewing system of claim 11, wherein said offset position of said proximal end is a distance at least as great as a diameter of said fitting.

15. The viewing system of claim 11, further comprising a computer processor operatively communicating with the image capture device, wherein said computer processor produces a plurality of control panel displays on the screen, said control panel displays comprising an image preview with a plurality of live subject views from said viewing instrument, an image control selector corresponding with an image control feature in said viewing instrument, and a capture image command for the image capture device to produce a captured image from at least one of said live subject views.

16. The viewing system of claim 15, wherein said computer processor is within the image capture device and receives a scope setting for said image control selector and further receives a corresponding instruction to perform a processing task, wherein said scope setting is selected from the group of settings consisting of a light intensity setting, a focus wheel setting, an aperture setting, a filter setting and any combination thereof, and wherein said processing task is selected from the group of tasks consisting of determining a focusing adjustment in said live subject views, calculating a differential power level between a focused image and an unfocused image, determining a refractive error from said live subject views, determining a calibration factor for optimizing said live subject views, performing a time countdown before starting to record said live subject views as a plurality of captured images, and any combination thereof.

17. The viewing system of claim 15, wherein said viewing instrument further comprises a plurality of sensors and a communications module, wherein said sensors measure a plurality of settings for a plurality of image control features in said viewing instrument, and wherein said communications module transmits said measured settings to said computer processor.

18. The viewing system of claim 17, wherein said computer processor is within the image capture device and receives a smart-scope selection for said image control selector option and further receives a corresponding instruction to perform a processing task, wherein said smart-scope selection instructs said communications module to automatically transmit said measured settings to said computer processor, wherein said measured settings are selected from the group of settings consisting of a light intensity setting, a focus wheel setting, an aperture setting, a filter setting and any combination thereof, and wherein said processing task is selected from the group of tasks consisting of determining a focusing adjustment in said live subject views, calculating a differential power level between a focused image and an unfocused image, determining a refractive error from said live subject views, determining a calibration factor for optimizing said live subject views, performing a time countdown before starting to record said live subject views as a plurality of captured images, and any combination thereof.

19. The viewing system of claim 18, wherein said viewing instrument further comprises a settings controller and wherein said control panel displays further comprise a setting selection option, wherein a setting selection entry to said setting selection option is transmitted from said computer processor to said settings controller and wherein said settings controller changes a scope setting for one of said image control features in said viewing instrument according to said setting selection entry.

20. The viewing system of claim 19, wherein said viewing instrument is an ophthalmoscope wherein said computer processor operatively communicates at least one of said measured controller settings and said captured images over a communications network.
